(19) United States
(12) Patent Application Publication
(10) Pub. No.: US 2012/0277714 A1
(43) Pub. Date: Nov. 1, 2012

(54) Designer underwear with special hidden absorbent pad wing support area

(76) Inventor: Risa Sophia Marcellus, Sacramento, CA (US)

(21) Appl. No.: 13/135,231

(22) Filed: Jun. 29, 2011

Related U.S. Application Data
(60) Provisional application No. 61/518,065, filed on Apr. 29, 2011.

(51) Int. Cl. A61F 13/72

(52) U.S. Cl. 604/396

(57) ABSTRACT

This invention relates to an item of underwear that is specifically worn in combination with sanitary pads, the kind that extend between the legs of the wearer over at least the pail of the crotch. Sanitary pads are for example, used by women during menstruation and are maintained in position by virtue of being adhered to the wearer's underwear by an adhesive strip and wings which are exposed by the removal of a protective sheet. The adhesive wings, which fold under the crotch of the underwear and are known to secure the pad into place, are normally visible.

This invention provides an additional strip of fabric mimicking the crotch of the underwear and attached onto and over the original crotch area which “CONCEALS” the adhesive wings that fold under the crotch of the underwear, making it so that the adhesive wings are not visible from the outside of the underwear.

This application claims benefits of the filing date of the provisional application No. 61/518065 Risa Sophia Marcellus Apr. 29, 2011, as the priority date of this non-provisional application.

According to the aspect of the invention, we at P3'S provide in combination an item of underwear including a front part, a back part, a waist part and a crotch part that is slightly wider in width, which in use extends front to back between the legs or a wearer, characterized in that the item of underwear includes an additional material lining part which extends over the length of the front of the original crotch part, to assist in “CONCEALING” the adhesive wings of a sanitary pad when used with this underwear invention.

We at P3'S have provided the invention of an additional strip or piece of fabric mimicking the crotch of the underwear, however, slightly thinner in width than the original crotch piece of material and is placed, sewn and attached onto and over the original crotch area, which is slightly wider, using a strong re-enforced cotton or spandex material of some kind. Having the material placed onto and over the original crotch area allows the wearer able to fold and tuck and “CONCEAL” the adhesive wings of any sanitary pad in between the original crotch material and the additional crotch material.

Thus the sides of the sanitary pad and its wings are “HIDDEN” or “CONCELED” between the original crotch part and the additional material lining part making a more comfortable confident arrangement for the wearer, meaning the side wings being located now tucked inside between the original lining of the crotch pail and the additional crotch material, and do not present unsightly visible white sanitary pad material in the crotch area when the wearer has on a short item of clothing or a item of swim wear or an item of intimate evening wear.

Preferably the additional lining part extends over the entire crotch part from front to back and perhaps also at least some of the front and back parts of the item or underwear.

For example, an item of underwear conventionally comprises a gusset provided by overlapping front and back pails, which gusset forms the crotch part of the garment. Thus the additional material lining pail may extend from front to back over a greater extent in width than such a conventional gusset pail so that sanitary pad may be attached to the additional material inside lining part over a substantial portion of its front and back, then its wings tucked and folded downward between its top additional material attached pail and its original pail.

The additional material lining part will comprise ends as well as open unattached sides. The sides may be shaped to conform generally to the sides of the crotch and each side will not extend outwardly beyond the sides of the original crotch part. The extra additional material lining pail will be attached to the remainder of the item of underwear along the ends done by triple stitching.

For comfort, preferably the carrier material is flexible, breathable, durable and machine washable.

For fashion the design will be, pretty & girly and lacey & sexy, sporty and casual swim bikini bottom wear. This will allow the wearer to use the item of underwear with extra bottom lining, during the monthly menstruation and still feel pretty and confident and sexy and sporty without revealing the otherwise obvious sanitary pad parts. The wearer will have more choices of style of clothing to wear during that special time of the month. The wearer will have the choice of using the sanitary pad instead of the tampon for a more natural, or comfortable, healthy, or religious choice.

### DRAWINGS

FIG. 1 is an illustrative view of an item of underwear with the combination in accordance with the invention;

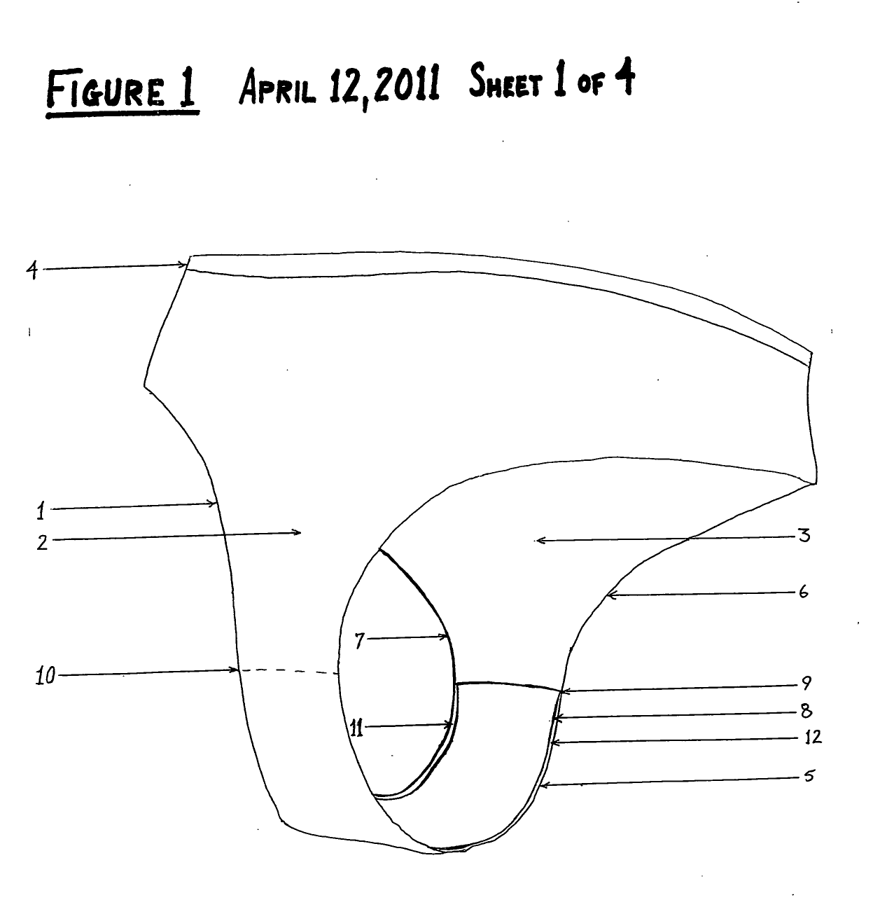

FIG. 2 is an illustrative view showing the item of underwear FIG. 1 in combination with a sanitary pad;

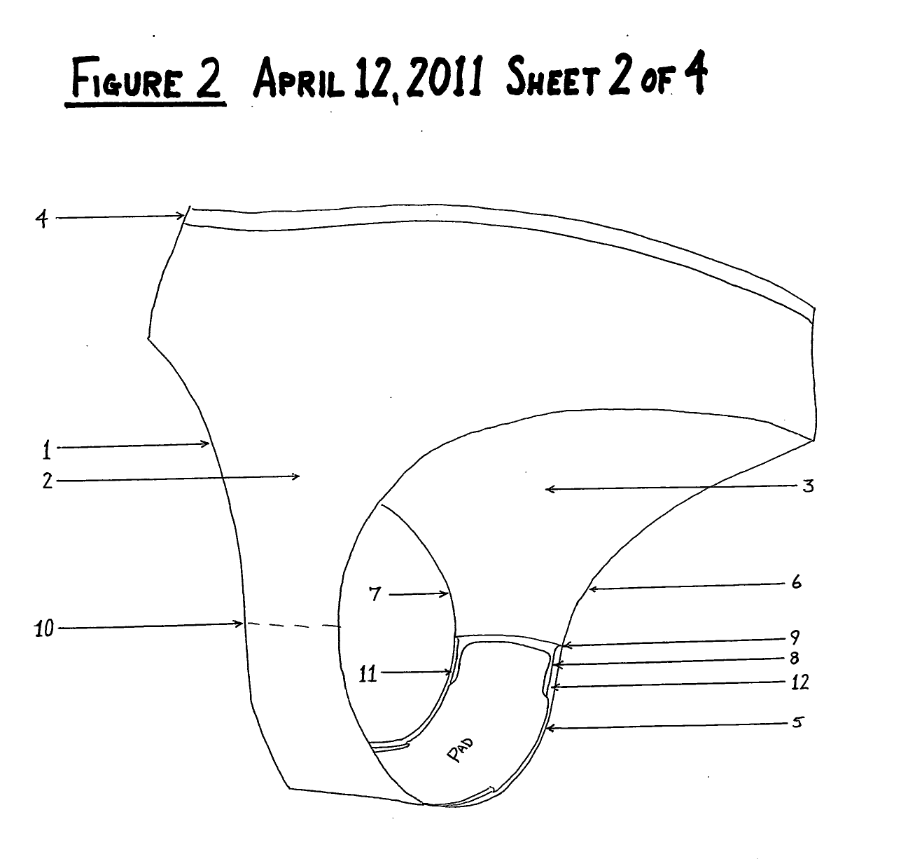

FIG. 3 is a more detailed plain view of the combination of FIG. 2.

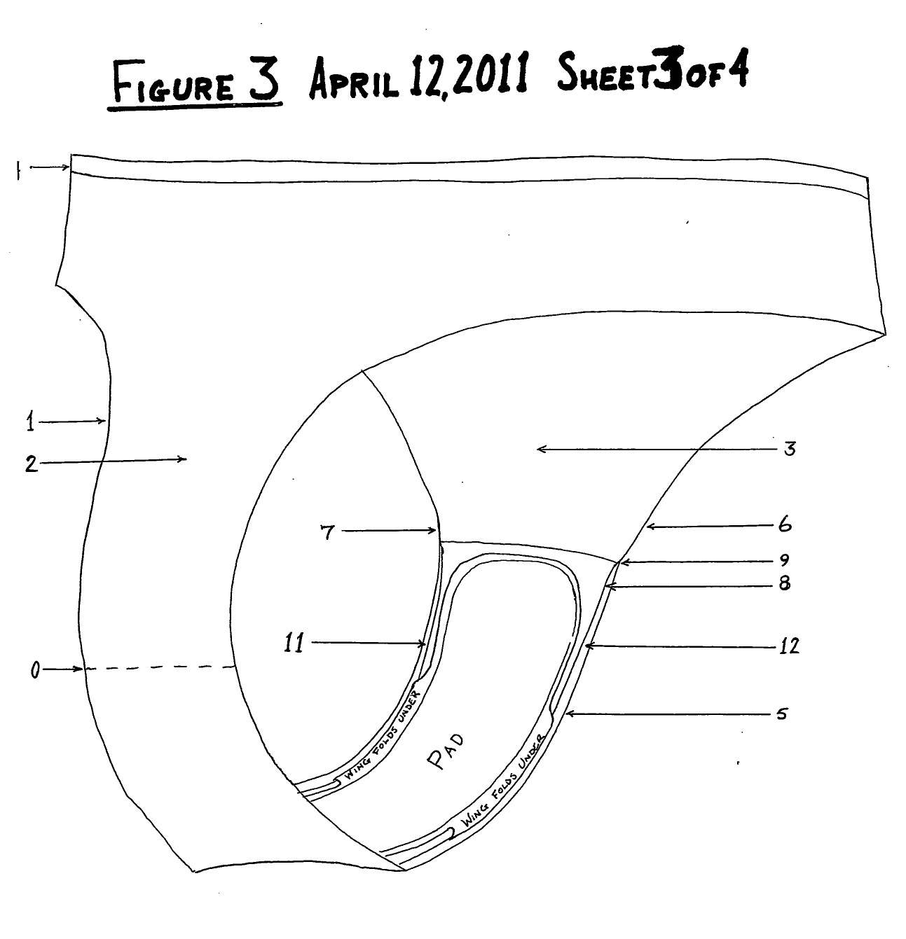

FIG. 4 are illustrative views of 3 drawings total, A) of the invention and of the inside view of the item of underwear's pail crotch in combination of the invention and sanitary pad, B) of the underside view of the item of underwear's pail and crotch without the invention and sanitary pad, C) of the underside view of the item of underwear's pail and crotch with one side of the sanitary wing in use and “concealed” with the invention and one side of the wing not in use with the invention nor concealed.

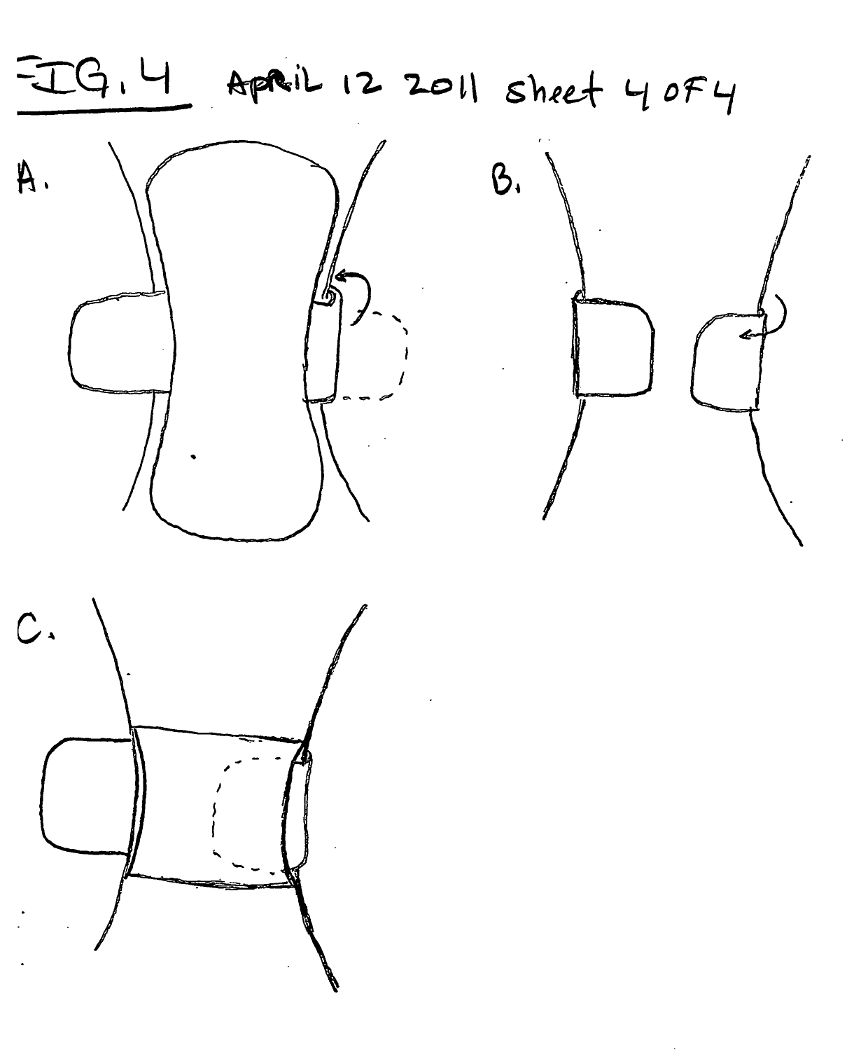

Referring to the drawings there is a shown item of underwear 1 comprising of a front part 2, a back part 3, which are connected to provide a waist part 4 and a crotch part 5, the crotch part 5 extending from front to back in use between a wearer's legs. The front 2 and back 3 parts provide openings 6, 7 for the wearer's legs and the crotch part 5 is provided by an elongated gusset of overlapping material of the front and back parts 2, 3.

In accordance with the invention there is an additional material lining part 8 which completely covers and extends over the top side of the original crotch length part 5 and extends to the front 2 and back 3 parts, where ends 9, 10 of the of the lining part are each connected by a lines of stitching, or can be one item of continuous material to the front 2 and back 3 parts. The additional material lining 8 has open sides 11 12 which extend front to back which are configured generally to conform to the original lining crotch part 5 but is slightly thinner on each side 11, 12 so the original crotch part 5 slightly larger ¼ of an inch or more than the additional material attached pail 8 sewn on top of the original crotch part 5. In this example, most portions of sides 11 12 of the additional material lining part 8 are unattached along at most of the length front to back for a purpose hereinafter explained.

In this example, where additional material top side lining is attached by double or more stitching 8 is open along the sides to allow the wearer the tuck down and fold the sanitary wings in between the original crotch part 5 and the attached additional material lining part 8 that is made of either cotton or spandex or combination material that is durable and breathable and machine washable.

What is claimed is:

1. In combination an item of underwear including a front part a back part, a waist part and a crotch part which in use extends front to back between a right leg and a left let of a wearer and a sanitary pad of a kind having side wings which carry an adhesive, wherein the item of underwear includes an inside lining part which extends over the length of the front of the original crotch part and has sides which are unattached to the crotch part over at least part of a front to back extent of the crotch part and the inside lining part further including a first end adjacent the front part and a second end adjacent the back part, said sides being attached to the underwear only at locations adjacent at least one of said first end and said second end, and the side wings of the sanitary pad are folded down into a position between the crotch additional lining material and the original crotch lining part with the adhesive wings taped underneath hidden out of sight in between the top additional lining material and the bottom original crotch lining part. When a sanitary pad is worn with the invention, the wings will not be seen or detected from the sides of the underwear, or below the bottom side of the underwear. This invention also prevents the wearer from getting stuck to various items and when sitting the invention provides protection from having the sticky wings stick to or touch the inner thighs of the wearer.

2. A combination according to claim 1 wherein the inside lining part extends over an entirety of the crotch part of the underwear from front to back.

3. A combination according to claim 1 wherein the crotch part includes crotch part sides, and the sides being shaped to conform generally to the crotch part sides but not outwardly beyond the crotch part sides, the inside lining part being attached to the item or underwear along the ends of the inside lining part.

4. A combination according to claim 3 wherein the inside lining part is attached to the item of underwear along parts of the sides of the inside lining part is unattached to the crotch part over at least a mid-portion of the crotch part.

5. A combination according to claim 1 wherein the inside lining part of the crotch has an additional lining material lining added on top of the original crotch lining part that is the same length but slightly smaller in width than the original crotch lining part.

6. A combination according to claim 5 wherein the additional crotch lining is made from some kind of cotton spandex blended material, for durability, flexibility and absorption and breathability and is machine washable.

7. An item of underwear including a front part, a back part, a waist part, and a crotch part which in use extends front to back between the legs of a wearer, and an inside lining part and an additional added lining material crotch part extends over the length of the front of the original crotch part and has sides which are unattached to the crotch part over at least part of the front to back extent of the crotch part and the inside lining part further including a first end adjacent the front part and a second end adjacent the back part, said sides being unattached to the underwear only at locations adjacent at least one of the said first end and said second end, a position between the crotch part and the inside lining part being adapted to receive side wings of a sanitary pad adhered below the top additional lining material and the original crotch lining part.
